(19) United States
(12) Patent Application Publication
(10) Pub. No.: US 2019/0117606 A1
(43) Pub. Date: Apr. 25, 2019

(54) COMPOSITIONS COMPRISING L-THEANINE, PROANTHOCYANIDIN/S AND A CATECHIN AND USES THEREOF

(71) Applicant: ALPHAGEN NZ LIMITED, Glendowie, Auckland (NZ)

(72) Inventors: Andrew Belton Scholey, Glendowie, Auckland (NZ); Angus Ross Brown, Glendowie, Auckland (NZ)

(21) Appl. No.: 16/301,811

(22) PCT Filed: May 11, 2017
(86) PCT No.: PCT/NZ2017/050058

§ 371 Date: Nov. 15, 2018

(30) Foreign Application Priority Data
May 17, 2016 (NZ) 720245

(51) Int. Cl. A61K 31/198; A61K 36/15; A61K 36/82; A61K 36/185; A61P 25/22; A61K 9/00; A61P 25/24; A61P 25/00; A61K 31/353; A23L 33/105; A23L 33/175; A23L 2/52

(52) U.S. Cl. CPC A61K 31/198; A61K 36/15; A61K 36/82; A61K 36/185; A61P 25/22; A61K 9/0056; A23V 2002/00; A61P 25/00; A61K 31/353; A23L 33/105; A23L 33/175; A23L 2/52; A61P 25/24

(57) ABSTRACT

The present invention relates to compositions including L-theanine, proanthocyanidin(s); and a catechin selected from the group consisting of epigallocatechin gallate, epigallocatechin, epicatechin gallate, epicatechin and combinations thereof. The compositions may be used to treat, prevent, or provide mental clarity following a period of time after consumption. The present invention also relates to methods of treatment, and methods of manufacture and use of said composition.

### TECHNICAL FIELD

The present invention relates to a functional composition and uses thereof, and in particular towards a composition that maintains or improves mental clarity.

### BACKGROUND ART

There is a growing need for humans to maintain or improve optimal mental clarity (as a component of a larger sub-set of attributes of overall cognitive performance), and/or deal with stress, in order to meet the demands of the ever-changing expectations of society. For example, there is increased social pressure and an expectation to deliver more efficiencies in the work environment, home environment, in the education system, and so forth.

In the context of the present invention, mental clarity should be considered the absence or suppression of distracting thoughts and feelings (i.e. internal distractions potentially caused by nervousness or uncertainty), the suppression of distracting or irrelevant sensory input (i.e. external distractions), together with reasonable level of vigour. This is compared to improved attention or cognitive performance, with the differentiation that a person may be attentive or have an improved level of “cognitive performance” but also be highly nervous or uncertain. Thus a person can be still very attentive but may not have mental clarity. Mental clarity is sought after because it allows people to remain calm and unstressed, but still be able to make rational decisions, remain concentrated and determined at a job at hand.

There is a huge and growing consumer demand for any medicament, functional food, herbal extract or supplement that helps individuals to achieve a high level of mental performance, but these can not necessarily achieve mental clarity. For example, many products include the likes of caffeine, which can often lead to a sense of jitteriness or nervousness, despite leading an apparent increase in cognitive function.

For example, Bayer markets a range of Berocca® products to support mental alertness.

Many foods or food supplements are also now heavily marketed towards improving or maintaining cognitive function, including various nuts, fruits and vegetables, fish oils, just to name a few. However, the downside with many of these foods is that they only provide a desirable effect after a sustained period of consumption, for example weeks or months, normally together with an overall healthy diet and lifestyle. Furthermore, it is often inconvenience or unpleasant to consume some food products in their unprocessed state. Supplements like fish oil capsules help to remedy this, but again such supplements are not seen by the public as having a desirable sensory perception profile.

The energy drink sector has enjoyed huge growth over recent years, based on health claims and marketing towards improving cognitive performance for a period of time after consuming the beverage, which also must taste good for commercial success. Most of these energy drinks rely on high levels of caffeine, and/or often have many unhealthy, or potentially harmful, ingredients in order to enhance the desired stimulatory effect or provide a beneficial taste. Furthermore, caffeine based energy drinks arguably may improve alertness to some extent, but as a side effect they can lead to a sense of hyper-activity, a loss of calmness, increased feeling of stress, a racing heart or a general uncomfortable jittery feeling, similar to the effects of having too much coffee. Furthermore, consuming too many of these energy drinks, either in a short time frame, or over an extended period, is a potential health concern.

Overall, there is a need to develop new functional foods/beverages that maintain or improve mental clarity without the need for caffeine, and to do so while also providing an associated calmness and lack of nervousness, rather than hyper-activity. Like-wise, there is a need to provide consumers functional foods/beverages that provide immediate effects on mental clarity soon after consumption, have a desirable taste and mouthfeel, offer convenience, and are generally healthy to consume.

Besides caffeine, there are a wide number of compounds and natural extracts that are thought to have some form of link to cognitive performance and/or mood/stress. For example, lemonbalm, Passionflower, Valerian, Sage, Guarana, Chamomile, Ashwagandha, Brahmi, roseroot, skullcap, iranin borage, gotu kola, ginkgo and milk thistle are believed to provide some promising attributes or felt cognitive effects.

Yet, it is unclear how such active ingredients or extracts might be able to work together to achieve desirable effects towards mental clarity.

To better understand and perhaps harness the potential effects associated with these ingredients, there has been significant research into a number of these chemical compounds or extracts as alternatives to caffeine.

However, success has been hampered either by a lack of Western based scientific assessment or human trials which are costly and time consuming, contradictory results between studies, poor flavour or stability profiles, and/or high cost of the active ingredients/manufacturing. Furthermore, some compounds or extracts may only provide no, or only a very slight, improvement, and therefore can often be viewed as not worth pursuing commercially.

It is also very difficult to predict or test results of altering the concentration/dosage of active ingredients to provide a desirable therapeutic effect. Also, research suggests that the associated effects of most compounds/extracts on cognitive performance, with mental clarity being only one aspect, appear to work through a variety of different complex modes of action, so it is very difficult to predict how different compounds may potentially work together if combined in attempt to provide a desired effect on improved mental clarity, if any.

Alternatively, the active agents may unintentionally negate one another, or provide unwanted results. Furthermore, combining different compounds together in attempt to provide synergies also can lead to potential issues with incompatibility/instability, or can negatively affect the sensory perception profile of a given composition. In summary, there is a significant hurdle of being able to study the effects of these different components, and it is impractical to test, and impossible to predict, different combinations of active agents for desirable health outcomes.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

It is an object of the present invention to address the foregoing problems or at least to provide the public with a useful choice.

Further aspects and advantages of the present invention will become apparent from the ensuing description which is given by way of example only.

### DISCLOSURE OF THE INVENTION

According to a first aspect of the present invention there is provided a composition including:
a) L-theanine;
b) proanthocyanidin(s); and
c) a catechin selected from the group consisting of epigallocatechin gallate, epigallocatechin, epicatechin gallate, epicatechin and combinations thereof.

According to a further aspect of the present invention there is provided a composition including:
a) L-theanine;
b) an extract from pine bark; and
c) an extract from Camellia sinensis or a green tea.

According to a further aspect of the present invention there is provided a use of a composition as herein described for the manufacture of a medicament for maintaining or improving mental clarity in a healthy individual.

According to a further aspect of the present invention there is provided a method of manufacturing a composition as herein described wherein the method includes the steps of mixing:
a) L-theanine or a source thereof;
b) proanthocyanidin(s) of a source thereof; and
c) a catechin selected from the group consisting of epigallocatechin gallate, epigallocatechin, epicatechin gallate, epicatechin and combinations thereof.

A method of supporting or improving mental clarity by administering to a person in need thereof with an effective amount of the composition as described herein.

### BRIEF SUMMARY OF THE INVENTION AND ITS ADVANTAGES

In a double-blind placebo-controlled randomized cross-over design in human subjects, the Applicant has surprisingly discovered that a trial composition according to the present invention led to an overall improvement in mental clarity after consumption, including:
a better ability to quickly dispense with distracting or misleading information;
a reduction of a sense of nervousness/tension/anxiety;
a reduction of a sense uncertainty/confusion/bewilderment;
an improved sense of vigour/liveliness/activity;
no feeling of increased fatigue.

These specific traits, in combination or even individually, are highly desirable and could not have been readily predicted based on the individual components used in the composition. First, there is a very wide number of potential active ingredients or extracts that could have been used in combination for trials, and it would have been impractical to do such testing to determine the effectiveness in a suitable double-blind cross over trial as provided. Furthermore, there was every likelihood that unexpected, unwanted effects could have been observed with the combination as trialed, including potentially cancelling out effects given the individual components themselves have a complex modes of action, which are yet to be fully understood.

Instead, the composition tested showed remarkable and unexpected results in terms of improved mental clarity. If fact, the inventors were actually expecting a different result in relation to improved cognitive performance, specifically around heightened memory, processing speeds, attention, and so forth. Such effects was not observed, but the unexpected mental clarity outcome seen was subsequently actually considered a more commercially important result.

Additionally, the composition has a good sensory perception, despite difficulties with poor taste or mouth feel of some individual components. Additionally, the product has good storage stability, which again was unpredictable given the significant number of active ingredients in the composition and the potential for incompatibility/interaction with the active agents. These advantages will be discussed further throughout the specification.

### DEFINITIONS AND PREFERRED EMBODIMENTS

The inventive composition may be formulated into a range of potential products. For example, the composition may be formulated as a liquid or semi-liquid beverage or as a food supplement such as a pill, capsule or powder. Alternatively, the composition may be formulated into a food item, such as a chocolate bar, or other similar snack. Most preferably, the composition is formulated as a beverage. Throughout the specification, the term mental clarity should be understood to include the absence, suppression or reduction of distracting thoughts and feelings (i.e. internal distractions potentially caused by tension or anxiety (e.g. nervousness) and/or confusion and bewilderment (e.g. uncertainty), the suppression or quicker identification of distracting, incorrect or irrelevant sensory input (i.e. external distractions), and/or an improved level of vigour. Mental clarity is a specific subset of overall mental performance, the latter which includes other factors such as composite memory, verbal memory, visual memory, processing speed, executive function, psychomotor speed, reaction time, complex attention, cognitive flexibility domains and overall neurocognitive index.

Preliminary tests (not shown) have indicated that the composition provides a significantly improved mental clarity for a period of about four hours after consumption compared to the control group. Further studies are being performed to assess these preliminary findings in more detail. The apparent synergistic effects as observed were also unexpected, given a potential contradiction with the actives, or inactivity when combined together. Furthermore, the heightened effects observed opens up opportunity to lower the dosage of one or more of the active agents to achieve a desirable result, which could help with lowering manufacturing costs, avoiding unwanted taste profiles of certain actives, and so forth.

L-Theanine

Throughout the specification, the term L-theanine should be understood to mean γ-glutamylethylamide, or a suitable source of L-theanine, such as a green tea extract (Camellia sinensis, or other species of Camellia) or the edible mushroom Xerocomus badius. Notably, the invention does not cover D-theanine. Synthetically derived L-theanine may also be used, or even L-theanine derivatives thereof, if shown to also provide the same functionality as described herein.

L-theanine is water soluble, known to cross the blood-brain barrier, and reaches peak concentrations about one hour after consumption. There is some scientific literature to support that L-theanine can have a relaxant effect, but can have some negative effects on other aspects of cognitive performance, especially if combined with other psychoactive agents. The inventors found that when L-theanine was combined with both proanthocyanidin and at least one catechin, a beneficial synergy was observed where trial participants developed a sense of improved overall mental clarity (e.g. including faster processing of incorrect cues, improved vigour, decreased nervousness and uncertainty) but without negative effects on overall cognitive performance.

Preferably, the composition includes at least 50 mg L-theanine.

Previous studies suggest that effects of L-theanine are observed at as little as 50 mg dosage; however the Applicant expects the beneficial effects will be seen between about 50 to 500 mg per dosage.

Most preferably, the composition includes about 50-200 mg L-theanine.

Preferably, the concentration of L-theanine is above 0.005% w/v, more preferably between 0.015 to 0.17% w/v, and most preferably about 0.08% w/v.

As described further below in the best modes section, these dosage amounts/concentration of L-theanine appeared to show the most promising results when in combination with the other components of the composition.

Of particular interest and surprise, the potential negative cognitive effects previously reported with L-theanine were not observed when combined with the other components of the composition. Equally, the calming effects of the L-theanine do not appear to have been diminished, and potentially have actually been amplified by the other components in combination.

Proanthocyanidin

Throughout the specification, the term proanthocyanidin should be understood to mean a class of polyphenols typically found in plants which are known to have powerful antioxidant properties. Proanthocyanidins are found in grapes, cranberry, apples, blueberries, and many other sources. A good source of proanthocyanidins is from pine bark extracts, from Pinus radiata (which is grown predominately in New Zealand) and Pinus pinaster, or Martime, pink bark (found predominantly in the Mediterranean region). Commercially available pine bark extracts (e.g. Enzogenol® or Pycnogenol®) which contain proanthocyanidins together with other condensed flavonoids have been shown to have therapeutic effects due to their powerful antioxidant activities.

Some preliminary studies have suggested beneficial links of proanthocyanidin to some factors associated with cognitive function, but it remains unclear whether the reported effects are due to increased blood flow, antioxidant action or other mechanisms. There is very little understanding of how these extracts may work if combined with other bioactives, or the potential effects on mood.

Here, the Applicant has shown that when pine bark extract is combined with L-theanine and EGCG, the test individuals showed a marked increase in mental clarity as previously discussed.

Preferably, the proanthocyanidin is provided within the composition as a pine bark extract.

More preferably, the proanthocyanidin is provided within the composition as Pinus radiata pine bark extract.

Preferably, the composition includes between 10 to 300 mg of proanthocyanidin or pine bark extract per dosage.

More preferably, the composition includes about 25 to 150 mg proanthocyanidin or pine bark extract per dosage. Most preferably the composition includes about 75 mg proanthocyanidin or pine bark extract per dosage

Preferably, the concentration of proanthocyanidin or pine bark extract in the composition is above 0.005% w/v, more preferably between 0.01 to 0.1% w/v, and most preferably about 0.06% w/v.

Although it is possible to exceed these amounts without any likely detrimental therapeutic effects, the pine bark extract can cause problems with the taste profile of the composition. However, the Applicant points out this can often be overcome by using masking agents, if required.

Catechin(s)

The four types of catechins found in green tea (from Camellia sinensis) are epigallocatechin gallate (EGCG), epigallocatechin (EGC), epicatechin gallate (ECG), and epicatechin (EC). EGCG accounts for about 50-80% of the catechins in green tea. Catechins may also be found from other sources such as cocoa.

Green tea and/or its bioactives (including L-theanine, caffeine and catechins as its major components) have been associated with many therapeutic effects, including cancer treatment, improved kidney function, diabetes, reducing hyperglycemia, reduction of cortisol production, reduction in inflammation, and cardiovascular health. However, further research needs to be conducted to clearly establish the modes of action and how they may potentially be used to therapeutically treat patients with these diseases. There is also some recent evidence to suggest catechins may impart some improvements in cognitive performance, but more understanding is required to establish a definitive mode of action or correlation.

Yet, the Applicant's research has found that when a green tea extract (and in particular, EGCG or other catechins) is combined specifically with L-theanine and proanthocyanidins, preliminary experiments suggest remarkable results in terms of improved mental clarity beyond which might have been expected from the individual components alone or when combined.

Preferably, the composition includes between 50 to 600 mg of EGCG or green tea extract per dosage.

More preferably, the composition includes about 100-300 mg EGCG or green tea extract per dosage.

Most preferably, the composition includes about 160 mg EGCG or green tea extract per dosage.

Preferably, the concentration of EGCG or green tea extract is above 0.005% w/v, more preferably between 0.01 to 0.5% w/v, and most preferably about 0.12% w/v.

Therefore, in one particularly preferred embodiment, the composition includes:
a) L-theanine between 0.015 to 0.17% w/v, and most preferably about 0.08% w/v;
b) a pine bark extract between 0.01 to 0.1% w/v, and most preferably about 0.06% w/v; and
c) a green tea extract between 0.01 to 0.5% w/v, and most preferably about 0.12% w/v.

In preliminary informal tests, working within these ratios and concentration was found to be particularly beneficial in providing the desired effects. Further trials are being performed to confirm the results seen.

Preferably, the composition also includes anthocyanin.

Anthocyanins are members of the flavonoid group of phytochemicals often present in berryfruit (amongst other foods), and are most often responsible for imparting a deep red/purple/blue pigment to the fruit.

The Applicant has also seen in preliminary trials that incorporation of berryfruit extracts, such as blackcurrant extract, appears to improve results even further. The Applicant envisages that the likely active ingredients is the anthocyanins; however it is unclear how these actives may be working synergistically with the other components in the composition. Interestingly, the Applicant has encountered negative flavour issues when the berryfruit is combined with the pine bark extract in particular. Although masking agents may be used to help address this issue, further work is being conducted to assess whether the undesirable flavour profile may be addressed through other avenues.

Preferably, the composition includes between about 100 mg to 500 mg berryfruit extract per dosage, or most preferably about 500 mg per dosage.

Preferably, the composition includes about 0.2% w/v berryfruit extract.

Preferably, the composition is caffeine free.

A significant advantage of the composition is that it does not rely on any need for a caffeine to provide the beneficial mental clarity effects, and avoids the jitteriness or anxiety associated with caffeine.

Preferably, the composition includes no added sugar.

In the context of a beverage, the composition may include a variety of excipients including natural flavours or fruit juice or concentrates; however a commercial advantage of the compositions developed is the lack of any need for added sugar to impart a pleasant tasting beverage.

Additionally, the developers also had to face self-imposed commercial goals to achieve target health ratings (including FSANZ regulations around the nutritional profiling scoring criterion) and also preferential Health Star ratings. Whilst this may seem trivial, it must be acknowledged that developing compositions with active ingredients such as these pose technical difficulty in achieving desirable shelf-life and active ingredient stability, important sensory perception profiles, health ratings, as well as achieving a functional cognitive benefit, namely mental clarity. The identification of the specific combination of active agents that not only is showing mental clarity, but also has a very good health profile (compliant with FSANZ Nutrient Profile Scoring Criterion), and allows commercially important stability and flavour profiles.

Uses/Treatments

Preferably, the composition is used to treat, prevent, maintain or improve mental clarity for a period of time after consumption. For instance, as illustrated by the preliminary data, the composition may be used to:
lower levels of tension/anxiety (or sub-sets of that such as nervousness); or
lower levels of confusion/bewilderment (or sub-sets of that such as uncertainty); or
improve levels of vigor activity (or sub-sets of that such as feeling lively, but without the jittery feeling often attributed from caffeine or other stimulants); or
prevent feelings of depression or fatigue;
or combinations thereof.

Alternatively the composition is used to treat, prevent, maintain or improve a healthy person's level of stress, mood, or sense of calmness.

### BEST MODES FOR CARRYING OUT THE INVENTION

### Brief Description of the Drawings

Further aspects of the present invention will become apparent from the ensuing description which is given by way of example only and with reference to the accompanying drawings in which:

FIG. 1. A schematic representation of the experimental timeline

FIG. 2. Effects of Placebo and Product on the Fatigue domain (composite of words Worn Out, Exhausted, Bushed, Weary and Fatigued). Error bars denote±SE.

FIG. 3. Effects of Placebo and Product on all POMS Domains (sub-scores). Error bars denote±SE.

FIG. 4. Effects of Placebo and Product on the word Lively (included in the Vigor-Activity domain). Error bars denote±SE.

FIG. 5. Effects of Placebo and Product on the word Nervous (included in the Tension-Anxiety domain).

Error bars denote±SE.

FIG. 6. Effects of Placebo and Product on the word Uncertain (included in the Confusion-Bewilderment domain). Error bars denote±SE.

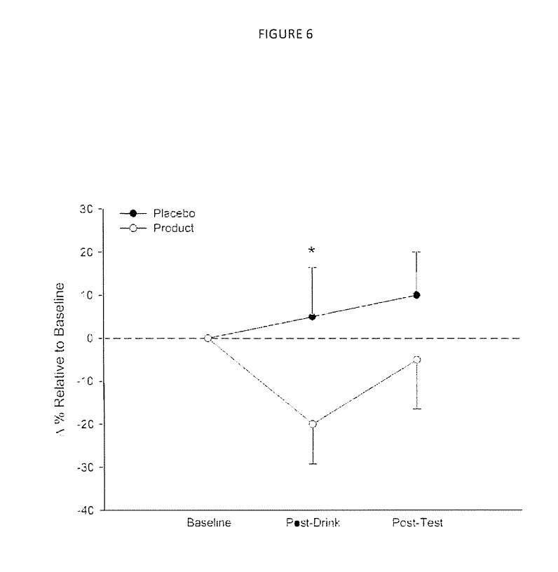

FIG. 7. Mean effect of Placebo and Product on reaction time to Endogenous cues. Error bars denote±SE. Panel A—Product*Cue Interaction.

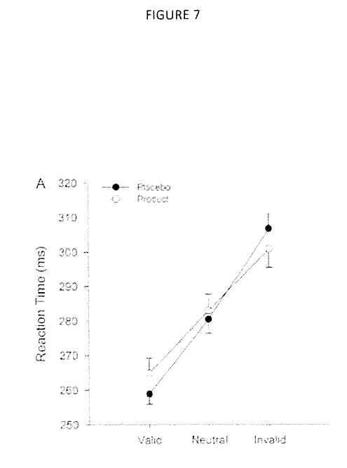

### EXAMPLE 1—BASE COMPOSITION

| | Preferred dosage | Preferred | |
|---|---|---|---|
| | range per 250 ml | dosage amount | |
| | serving (and ratios | per 250 ml | Preferred |
| Component | derived therefrom) | serving | concentration |
| L-theanine | 50 mg to 500 mg | 200 mg | 0.08% w/v |
| Pine bark extract, | 50 mg to 300 mg | 150 mg | 0.06% w/v |
| Pinus Radiata | | | |
| EGCG | 100 mg to 700 mg | 300 mg | 0.12% w/v |

### EXAMPLE 2—FUNCTIONAL BEVERAGE CONTAINING BASE COMPOSITION

| Name | Quantity | UOM |
|---|---|---|
| Organic Apple Juice Conc | 134.82 | Grams |
| Blackcurrant Juice Conc | 8.81 | Grams |
| Citric Acid | 1 | Grams |
| Xanthan Gum | 1 | Grams |
| Green tea extract (98% EGCG) | 1.2 | Grams |
| L-theanine | 0.8 | Grams |
| Pinus radiata bark extract | 0.6 | Grams |
| (Enzogenol ®) | | |
| Flavouring | 4.9 | Millilitres |
| Water to volume | 1 | Litre |

### EXAMPLE 3—FUNCTIONAL BEVERAGE CONTAINING BASE COMPOSITION AND BLACKCURRANT EXTRACT

| Name | Quantity | UOM |
|---|---|---|
| Organic Apple Juice Conc | 134.82 | Grams |
| Blackcurrant Juice Conc | 8.81 | Grams |
| Citric Acid | 1 | Grams |
| Xanthan Gum | 1 | Grams |
| Blackcurrant Extract | 2 | Grams |
| Green tea extract (98% EGCG) | 1.2 | Grams |
| L-theanine | 0.8 | Grams |
| Pinus radiata bark extract | 0.6 | Grams |
| (Enzogenol ®) | | |
| Flavouring | 4.9 | Millilitres |
| Water to volume | 1 | Litre |

### EXAMPLE 4: DOUBLE-BLIND PLACEBO CONTROLLED CROSS OVER STUDY IN HUMANS

### Summary

The aim of this project was to explore effects of a plant extract beverage on cognitive performance. Functional ingredients within the formulation included extracts of pine bark, L-theanine and a green tea leaf extract. Several small human trials have reported acute changes in cognition following ingestion of other extracts, but the unique combination and dose used in the current studies are untested.

The plant extracts (or a cellulose placebo) were ingested in capsule form because the distinctive taste qualities of these extracts made it challenging to formulate a taste-matched placebo solution. Each treatment was ingested alongside 250 ml of the product's juice base. Participants underwent a mood assessment three times per trial, visual tests twice, and neurocognitive testing 30 minutes after consuming each test drink+capsules.

Unexpectedly, the test beverage had no detectable improvement effects on overall cognitive performance measured via the cognitive test battery. Yet, beneficially, there was no decrease in cognitive performance. However, also unexpectedly, there was a dramatic effect on mental clarity, and in particular an ability to process invalid ques, an increase in the vigour-activity domain, a decrease in anxiety domain, and/or a decrease in the confusion-bewilderment domain, particularly in the post-drink and extending somewhat after post-test time-points. These unexpected attributes are seen to be a major commercially important outcome. For example, consumers may feel a sense of vigour without the nervous/jitteriness feelings associated with other drinks such as those containing caffeine. Similarly, with the lack of nervousness and uncertainty, decision making and clear-headedness is what may be contributing to the faster ability to process invalid cues.

Interestingly, higher levels of fatigue were observed post-test in the product vs placebo groups when using the POMS-SF inventory. However, no differences were observed between test drinks with visual analogue fatigue response scale, which generally is given greater weighting than the POMS-SF for fatigue assessment.

Method

A Product and Placebo treatment were administered within a double-blind placebo-controlled randomised crossover design. Healthy adults visited the laboratory on two occasions, each visit was separated by 7 days. The trial order was block randomised with participants has equal probability of receiving both treatment orders. A fixed block size was used for stratification of participant sex.

20 healthy volunteers took part in the study (11 Female, 9 Male), with a mean age of 24 years (range 31-19), mass of 73 kg±14 and height of 1.8 m±0.1. The occupational distribution of the participants was 55% professionals and 45% students. Testing sessions were separated by a mean of 7 days (range 5-9 days).

On each visit to the laboratory participants ingested 2 capsules with 250 ml of the product juice base packaged in the product bottle. Capsules contained either plant extracts or cellulose. This approach was taken because the distinctive taste qualities of the plant extracts made it challenging for the funder to formulate a taste-matched placebo solution within appropriate sensory discrimination thresholds.

The Product capsules contained 75 mg of pine bark extract (Enzogenol) and 160 mg of green tea extracts; min. 94% EGCG) and 100 mg L-theanine. The Placebo capsules contained 335 mg of a cellulose filler. All ingredients were licensed by NZ Food Safety authorities and will be combined and packaged in a licensed commercial facility (ENZO Nutraceuticals Quality Control Laboratory). Capsules and the fruit juice base were stored at 4° C. and consumed within a research kitchen (University of Auckland, Building 731.120, Tamaki Campus).

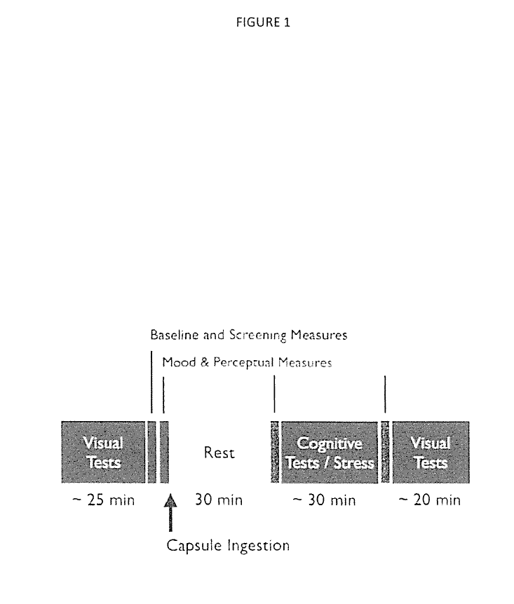

Thirty minutes after consuming the drink participants performed a standardised computer-based assessment of cognition and vision. A schematic representation of the experimental timeline is shown in FIG. 1.

The cognitive test battery comprised seven tests: verbal and visual memory, finger tapping, symbol digit coding, the Stroop test, a test of shifting attention, and a continuous performance test. A detailed description of each test is given by Gualtieri and Johnson (2006). Standardised computer instructions on how to complete each test were given before the test along with practice sessions when necessary. Tests were conducted in an environmentally-controlled and sound-proofed chamber. Tests were initiated and supervised (via a viewing window) by an experimenter and delivered unassisted and uninterrupted.

Test scoring was generated from 17 primary scores based on correct responses, error responses, number of responses, and reaction times. Primary scores were used to generate nine neurocognitive domain scores to reflect basic mental functions (composite memory, verbal memory, visual memory, processing speed, executive function, psychomotor speed, reaction time, complex attention, and cognitive flexibility) and an overall neurocognitive index score. Domain scores were generated as raw scores, calculated from composite primary scores of relevant tests, and then computed to standard scores and percentiles that represent the participants' raw score relative to an age-matched normative data set of healthy individuals. Standardized domain scores and the 17 primary scores were used for statistical analysis.

Upon completion of the test battery, a six-point Likert-type rating scale was presented on the computer screen prompting the participant to rate their current level of alertness ranging from 1 (exhausted, unable to function effectively) to 6 (fully awake and excellent alertness).

The POMS-SF is inventory that measures psychological distress via mood disturbance scores using a 30 word list of words. Each adjective is rated from 0-4 in terms of how the participant feels in the present moment; 0—not at all, 1—a little, 2—moderately, 3—quite a bit, and 4—extremely. Each word falls under one of five domains; Depression, Vigor-Activity, Tension-Anxiety, Confusion-Bewilderment, or Fatigue. The scores given to the words that fall within each domain are then combined for an overall score. The POMS-SF was administered three times during the protocol, at Baseline, 30 min Post-Drink, and Post-Test, along with the perceptual scales.

Covert spatial attention was assessed using a visual cueing task. The initial baseline display consisted of a central fixation cross and two peripheral boxes. The instruction was to maintain fixated on the central cross at all times while responding as quickly and accurately as possible upon perception of a circular target appearing within one of the peripheral boxes. Participants responded using the left or right arrow keys on the keyboard. Before the appearance of the target, a cue was presented for 200 ms. The cue was either Endogenous (an arrow pointing to the right or left box), or Exogenous (an increase in the line width of the left or right box). Valid trials consisted of the target appearing in the cued direction, whereas for Invalid trials the target appeared opposite to the cued location. Neutral trials gave no indication as to where the target may appear, and were presented as a double-ended arrow in the endogenous trials, and an increase in the line width for both boxes in the exogenous trials. In total, 180 trials were collected, split evenly between endogenous and exogenous cues. Valid, invalid and neutral trials were then randomised within the endogenous and exogenous blocks. Keyboard responses were collected using customised Matlab software (MathWorks R2010b, Massachusettes, USA). To ensure that participants maintained fixation on the central cross, eye movements were monitored with a head-fixed eye tracking system (ViewPoint Eye Tracker, Arrington Research Systems, Scottsdale, USA). Any trials in which the eyes deviated were rejected from analysis. Participants completed the covert attention task at baseline and again Post-Test, after the cognitive test battery.

Analysis

All self-assessments and perceptual scales were converted into a percentage along the continuum on which they were rated. Motivation, diet and stress were compared between the two sessions using a Student's paired t-test. A 2 Treatment×3 Time Repeated Measures ANOVA with SPSS was used to assess changes in the perceptual scales, as well as the POMS questionnaire. Analysis was conducted for the five POMS domains, as well as on each individual word that contributed to the domain scores. Whilst the raw data was used for statistical analysis, the Post-Drink and Post-Test results have been normalised relative to the baseline measure for graphical representation.

Paired t-tests were used to compare the time taken to complete the cognitive test battery, alertness rating score, and all results for each individual test and subsequent domains. A missing data analysis was required for one missing data point in the alertness rating score for a participant in their Product trial. This took the mean value for the rest of the group, rounded to the nearest whole number.

Reaction Time and Percentage of Correct responses in the Covert Attention task were split into Endogenous or Exogenous categories, and each analysed using a 2 Treatment×2 Time×3 Cue Type RM ANOVA. Validity scores were then calculated by subtracting the Valid RT from the Invalid, and compared using a 2 Treatment×2 Time RM ANOVA. Statistical significance was set at p≤0.05 and all means are reported±SE.

Results

Readiness to Participate

Participants rated their motivation to participate in the trials as 76%±1.7 on a scale from very bad to very good. They evaluated their diet over the 24 hours prior to each trial as 61%±2.8 on a scale from very bad to very good, and their stress levels over the last 24 hours as 48%±3.2 on a scale from very bad to very good in relation to normal. There were no differences between treatments or effects of trial order on these variables.

Perceptual Ratings

There was a significant effect of time (p=0.001) across both treatments for “Feeling”; when asked what type of mood they were in, on a scale from very bad to very good. Evaluations of Feeling tended to increase Post-Drink (although this change was not significant) and then decreased below baseline values Post-Test. There was no difference between the treatments. When evaluating Arousal from low to high, and Fatigue from not fatigued to highly fatigued, there were no effects of treatment, time or trial order.

Mood Disturbance Ratings (POMS-SF)

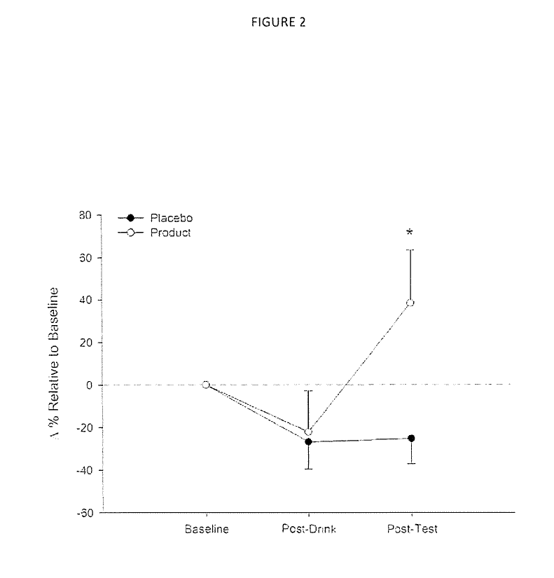

There was a significant interaction between treatment and time for the POMS domain of Fatigue (p=0.01) (FIG. 2), as well as a main effect of time (p=0.003).

Both drinks resulted in a decrease in ratings of fatigue, however this decrease was only maintained in the placebo trial, whereas the product resulted in an increase above baseline values (post-test). We believe this effect may merely because the product group was exerting more effort during the test. Of the 20 participants, 13 expressed higher levels of fatigue in the product treatment, compared with the placebo. Further investigation into the specific words used in the POMS questionnaire showed that this result was driven by significant interactions for the words Exhausted (p=0.026) and Bushed (p=0.022). We also note that other tests (visual analogue psychometric response scale) for fatigue showed no difference between the test groups.

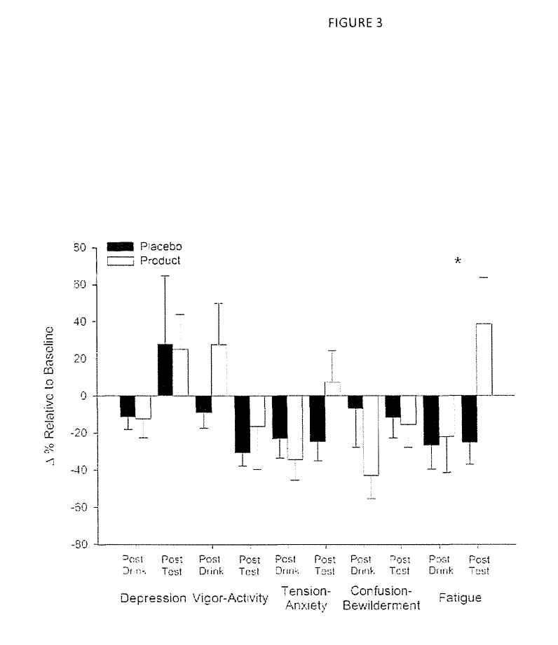

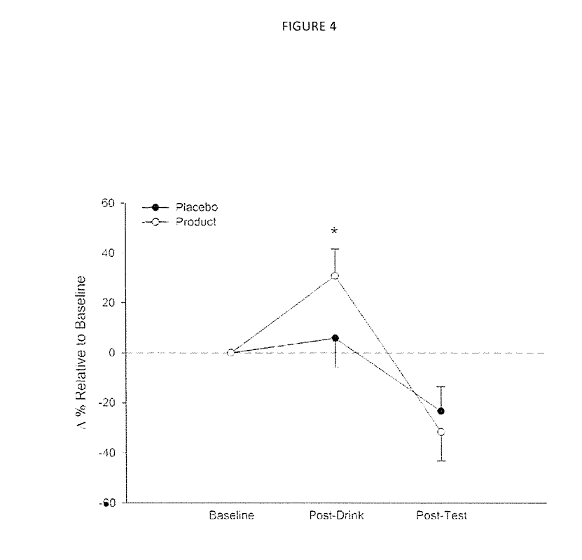

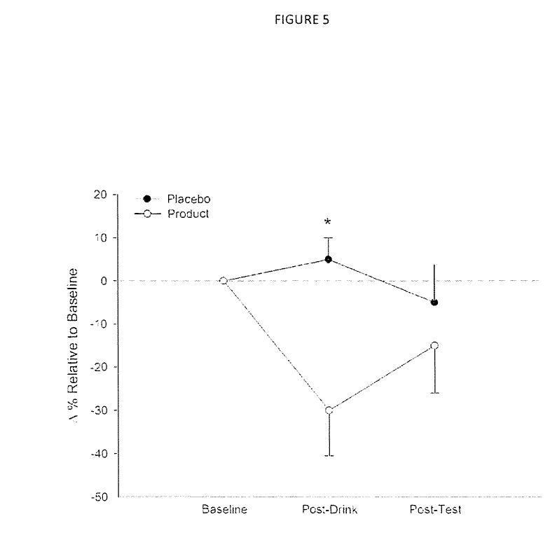

Looking at FIG. 3, post-drink results showed significant improvement in vigor-activity domains in the product group, lower tension-anxiety and confusion-bewilderment in the product group compared to the placebo. There were interactions between treatment and time for the words Lively, from the Vigor-Activity domain (p=0.025) (FIG. 3), and Nervous, from the Tension-Anxiety domain (p=0.034) (FIG. 4) and a main effect of treatment for Uncertain, from the Confusion-Bewilderment domain (p=0.045) (FIG. 5).

Cognitive Performance

Participants spent on average 28 min 11 s±46.5 s completing the cognitive test battery, with no differences across treatment or trial order. There were no effects of treatment on any cognitive domains (Table 1), or individual test results. Alertness was rated on a scale with the associated labels; 1—Exhausted, unable to function effectively, 2—Sleepy but able to function, 3—Tired but able to function, 4—Awake, able to function effectively, 5—Awake and mentally alert, 6—Fully awake and excellent alertness. A mean of 3.4±0.14 was reported, with no differences with treatment or trial.

TABLE 1
Effects of the treatments on cognitive domain
scores. Data show the group mean ± SE.

| | | | | | Effect of |
|---|---|---|---|---|---|
| | Placebo | | Product | | Product |
| | Mean | SE | Mean | SE | p |
| Neurocognitive index | 115.95 | 3.22 | 115.60 | 2.47 | 0.87 |
| Composite memory | 111.70 | 2.86 | 110.40 | 2.76 | 0.70 |
| Verbal memory | 110.45 | 3.11 | 108.00 | 3.73 | 0.51 |
| Visual memory | 109.00 | 3.26 | 109.20 | 2.51 | 0.96 |
| Processing speed | 169.10 | 7.15 | 175.45 | 6.07 | 0.38 |
| Executive function | 118.15 | 4.39 | 115.35 | 4.77 | 0.45 |
| Psychomotor speed | 142.60 | 4.93 | 145.65 | 3.76 | 0.41 |
| Reaction time | 114.30 | 2.96 | 113.65 | 3.02 | 0.72 |
| Complex attention | 96.20 | 5.73 | 96.30 | 4.22 | 0.99 |
| Cognitive flexibility | 120.55 | 6.07 | 113.30 | 5.10 | 0.32 |

Note:
Standard scores have a normative mean of 100 and SD of 10. Qualitative labels associated with performance levels include “very low” (<70), “low” (70-79), “low-average” (80-89), “average (90-109) and “above average” (>109). Data were compared between treatments using a paired t---test, and statistical significance was set at p ≤ 0.05.

Covert Attention Task

For the Endogenous trials, there was a significant effect in that the product group was able to process and identify invalid cues significantly quicker than the placebo group. Additionally, overall there was a smaller difference in reaction times between valid and invalid cues for the product compared to the placebo. This may possibly indicate that in the product trials it was easier to disengage covert attention from the invalid cues and direct to the target, hence pointing to a more even decision making progress during the product trial. We believe this is a beneficial outcome providing more balanced decision making, aligned with the calming, lower nervousness effects provided by the product.

Interpretation

Overall, the trial unexpectedly did not result in an improved overall cognitive performance. However, it did substantially improve a key aspect of cognitive performance, including mental clarity/mood, and/or being able to process invalid cues quicker. These were very unexpected results, and which have considerable commercial implications.

The trial showed that the product group had significant improvements in key domains post-drink (vigour-activity, tension-anxiety, and confusion-bewilderment), and these results were particularly emphasized at the post-drink time point. At the same time, no negative effects were observed for depression or fatigue POMS Domains. Commercially speaking, post-drink outcomes are considered to be more important than post-test outcomes as the post-drink is considered to be what primes the consumer to perform their daily tasks after consumption and act with mental clarity. Furthermore, the product group also importantly showed greater ability to quickly dispense with invalid cues, and be more balanced in terms of time taken to assess valid and invalid cues, both considered to be attributes of mental clarity.

The mechanisms of actions for each herbal ingredient is not fully understood, including any interactions or synergies between extracts, or components of the juice base.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

What I/We claim is:

1. A composition including:
a) L-theanine;
b) proanthocyanidin(s); and
c) a catechin selected from the group consisting of epigallocatechin gallate, epigallocatechin, epicatechin gallate, epicatechin and combinations thereof.

2. A composition including:
a) L-theanine;
b) an extract from pine bark containing proanthocyanidin(s); and
c) an extract from Camellia sinensis or a green tea containing a catechin selected from the group consisting of epigallocatechin gallate, epigallocatechin, epicatechin gallate, epicatechin and combinations thereof.

3. The composition as claimed in any one of the above claims wherein the composition includes between about 50 mg to 200 mg L-theanine.

4. The composition as claimed in any one of the above claims wherein the concentration of L-theanine is between about 0.015 to 0.17% w/v.

5. The composition as claimed in any one of the above claims wherein the proanthocyanidin or extract from pine bark in the composition is between about 10 to 300 mg.

6. The composition as claimed in any one of the above claims wherein the concentration of proanthocyanidin or pine bark extract in the composition is between about 0.01 to 0.1% w/v.

7. The composition as claimed in any one of the above claims wherein the proanthocyanidin in the composition is an extract from Pinus radiata pine bark.

8. The composition as claimed in any one of the above claims wherein the composition includes between about 50 to 600 mg of EGCG or green tea extract.

9. The composition as claimed in any one of the above claims wherein the concentration of EGCG or green tea extract is between about 0.01 to 0.5% w/v.

10. The composition as claimed in any one of the above claims wherein the composition also includes an anthocyanin, or a berryfruit extract containing anthocyanin.

11. The composition as claimed in claim 10 wherein the berryfruit extract is a blackcurrant extract.

12. The composition as claimed in claim 10 or 11 wherein the composition includes between about 100 mg to 500 mg or anthocyanin or berryfruit extract containing anthocyanin.

13. The composition as claimed in claims 10 to 12 wherein the composition includes about 0.2% w/v berryfruit extract.

14. The composition as claimed in any one of the above claims wherein the composition is caffeine-free and/or sugar-free.

15. The composition as claimed in any one of the above claims wherein the composition is formulated as a liquid or semi-liquid beverage, a food or food supplement, a pill, capsule or powder.

16. A method of treatment to support, maintain, prevent or improve
a) mental clarity,
b) tension/anxiety,
c) confusion/bewilderment,
d) vigor activity,
e) depression or fatigue
f) or combinations thereof
by administering to a person in need thereof with an effective amount of the composition as claimed in any of claims 1 to 14.

17. A use of a composition as claimed in any one of claims 1 to 14 in the manufacture of a medicament for supporting, maintaining, preventing or improving
a) mental clarity,
b) tension/anxiety,
c) confusion/bewilderment,
d) vigor activity,
e) depression or fatigue
f) or combinations thereof.

18. A method of manufacturing a composition as herein described wherein the method includes the steps of mixing:
a) L-theanine or a source thereof;
b) proanthocyanidin(s) or a source thereof; and
c) a catechin selected from the group consisting of epigallocatechin gallate, epigallocatechin, epicatechin gallate, epicatechin and combinations thereof.
